(19) United States
(12) Patent Application Publication
(10) Pub. No.: US 2026/0317213 A1
(43) Pub. Date: Oct. 15, 2026

(54) METHOD FOR MONITORING DISSOLVED OXYGEN IN BIOFILM, OXYGEN SUPPLY OPTIMIZATION METHOD AND SYSTEM, AND DEVICE

(71) Applicant: Zhejiang University, Hangzhou City (CN)

(72) Inventors: Liang Zhu, Hangzhou City (CN); Chisheng Yu, Hangzhou City (CN); Zhiwei Liang, Hangzhou City (CN); Zhuodong Yu, Hangzhou City (CN); Tianyu Xu, Hangzhou City (CN)

(73) Assignee: Zhejiang University, Hangzhou City, ZJ (CN)

(21) Appl. No.: 19/343,326

(22) Filed: Sep. 29, 2025

(30) Foreign Application Priority Data
Apr. 9, 2025 (CN) 202510435913.4

(51) Int. Cl. C02F 3/00; C02F 3/12; C02F 3/20; G01N 33/18; G06V 10/44; G06V 10/56; G06V 10/70; G06V 20/52

(52) U.S. Cl. CPC C02F 3/006; C02F 3/1268; C02F 3/208; G01N 33/18; G06V 10/44; G06V 10/56; G06V 10/70; G06V 20/52; C02F 2209/006; C02F 2209/08; C02F 2209/14; C02F 2209/16; C02F 2209/22; C02F 2209/38

(57) ABSTRACT

Provided are a method for monitoring dissolved oxygen (DO) in a biofilm, an oxygen supply optimization method and system, and a device, relating to the technical field of wastewater treatment. The monitoring method includes: acquiring image data of a biofilm in a Membrane Aerated Biofilm Reactor (MABR) wastewater treatment apparatus during wastewater treatment; performing feature extraction on the image data to obtain image feature parameters; and inputting the image feature parameters into a biofilm DO prediction model to obtain an effective DO concentration within the biofilm outputted by the biofilm DO prediction model, where the biofilm DO prediction model is trained based on a machine learning model. This application provides a method for monitoring DO in a biofilm using machine learning-based image recognition, overcoming the drawbacks of microelectrodes such as high cost, susceptibility to damage, and difficult maintenance, thereby achieving low-cost and effective monitoring of DO in biofilms.

### CROSS REFERENCE TO RELATED APPLICATION

This patent application claims the benefit and priority of Chinese Patent Application No. 202510435913.4, filed with the China National Intellectual Property Administration on Apr. 9, 2025, the disclosure of which is incorporated by reference herein in its entirety as part of the present application.

### TECHNICAL FIELD

The present disclosure relates to the technical field of wastewater treatment, and in particular, to a method for monitoring dissolved oxygen (DO) in a biofilm, an oxygen supply optimization method and system, and a device.

### BACKGROUND

Membrane Aerated Biofilm Reactor (MABR) is an innovative wastewater treatment process that combines gas separation membrane technology with biofilm-based wastewater treatment technology. This process utilizes MABR as a microbial carrier and provides bubble-free aeration for microorganisms, creating aerobic, anoxic, and anaerobic biological environments from the membrane layer outward, thereby achieving simultaneous nitrogen removal and organic matter degradation. Compared with traditional aeration processes, MABR offers advantages such as low energy consumption, reduced biofilm detachment, and high oxygen utilization efficiency.

In MABR, oxygen is supplied from the lumen of membrane fibers and diffuses through the membrane into the biofilm. As a result, the dissolved oxygen (DO) concentration in the biofilm decreases from an inner layer of the biofilm (near the membrane) toward a biofilm-liquid boundary layer along a biofilm thickness direction. Meanwhile, nutrients and other pollutants diffuse from bulk liquids into the biofilm. Unlike conventional biofilm reactors with co-diffusion biofilms, electron acceptors (such as nitrate and nitrite) and electron donors (such as ammonia nitrogen and chemical oxygen demand) diffuse into the MABR biofilm from opposite directions, forming a unique counter-diffusion biofilm.

Since oxygen diffuses from the interior, monitoring DO in the solution cannot accurately reflect the oxygen conditions within the biofilm. Even if the DO in the solution is zero, the biofilm can still achieve effective water purification due to the formation of a biotic environment with an inner aerobic layer and an outer anaerobic layer. Although microelectrode technology can be used to monitor DO within the biofilm, microelectrodes are costly, prone to damage, and difficult to maintain, making them impractical for real-world applications.

### SUMMARY

An objective of the present disclosure is to provide a method for monitoring DO in a biofilm, an oxygen supply optimization method and system, and a device, overcoming the drawbacks of microelectrodes such as high cost, susceptibility to damage, and difficult maintenance, thereby achieving low-cost and effective monitoring of DO in biofilms.

To achieve the above objective, the present disclosure provides the following technical solutions.

According to a first aspect, the present disclosure provides a method for monitoring DO in a biofilm, where the method for monitoring DO in a biofilm is applied to an MABR wastewater treatment apparatus, and includes:
acquiring image data of a biofilm in the MABR wastewater treatment apparatus during wastewater treatment;
performing feature extraction on the image data to obtain image feature parameters; and
inputting the image feature parameters into a biofilm DO prediction model to obtain an effective DO concentration within the biofilm outputted by the biofilm DO prediction model, where the biofilm DO prediction model is trained based on a machine learning model.

Optionally, the image feature parameters include: biofilm surface bubble density, surface bubble size, biofilm thickness, biofilm color, and biofilm temperature distribution.

According to a second aspect, the present disclosure provides an oxygen supply optimization method based on image recognition, where the oxygen supply optimization method based on image recognition is applied to an MABR wastewater treatment apparatus, and includes:
acquiring image data of a biofilm in the MABR wastewater treatment apparatus and water quality monitoring parameters in a biochemical reaction unit of the MABR wastewater treatment apparatus during wastewater treatment;
performing feature extraction on the image data to obtain image feature parameters;
inputting the image feature parameters into a biofilm DO prediction model to obtain an effective DO concentration within the biofilm outputted by the biofilm DO prediction model, where the biofilm DO prediction model is trained based on a machine learning model; and generating an oxygen supply control signal based on the effective DO concentration and the water quality monitoring parameters, and adjusting an oxygen supply amount of a gas delivery unit in the MABR wastewater treatment apparatus.

Optionally, the water quality monitoring parameters include: influent ammonia nitrogen, effluent ammonia nitrogen, total nitrogen, chemical oxygen demand (COD) concentration, and DO concentration in the biochemical reaction unit; and the image feature parameters include: biofilm surface bubble density, surface bubble size, biofilm thickness, biofilm color, and biofilm temperature distribution.

Optionally, the adjusting the oxygen supply amount of the gas delivery unit in the MABR wastewater treatment apparatus includes one or more of: adjusting an aeration pressure, adjusting a gas flow rate, and adjusting an aeration mode.

According to a third aspect, the present disclosure provides an oxygen supply optimization system based on image recognition, where the oxygen supply optimization system based on image recognition is applied to an MABR wastewater treatment apparatus, and includes: an oxygen supply adjustment apparatus, an image recognition unit, a DO monitoring apparatus, and an intelligent terminal;
where the oxygen supply adjustment apparatus is disposed between a gas delivery unit of the MABR wastewater treatment apparatus and a heterogeneous mass transfer biofilm membrane module;
the image recognition unit and the DO monitoring apparatus are arranged in a biochemical reaction unit of the MABR wastewater treatment apparatus; the image recognition unit and the DO monitoring apparatus are both connected to the intelligent terminal, and the intelligent terminal is connected to a control end of the oxygen supply adjustment apparatus;
the image recognition unit is configured to acquire image data of a biofilm in the heterogeneous mass transfer biofilm membrane module, and the DO monitoring apparatus is configured to obtain water quality monitoring parameters; and
the intelligent terminal is configured to obtain an oxygen supply control signal based on the image data and the water quality monitoring parameters by using the above oxygen supply optimization method based on image recognition, and control the oxygen supply adjustment apparatus according to the oxygen supply control signal to regulate oxygen distribution.

Optionally, the oxygen supply adjustment apparatus includes one or both of a membrane gas supply pressure monitoring component and a flow rate adjustment component.

Optionally, the image data is acquired by one or more of: an underwater camera, a laser scanning imager, and an infrared camera.

Optionally, a distance between the image recognition unit and the heterogeneous mass transfer biofilm membrane module is 30 mm to 1000 mm; and a distance between the DO monitoring apparatus and the heterogeneous mass transfer biofilm membrane module is 10 mm to 30 mm.

According to a fourth aspect, the present disclosure provides computer device, including a memory, a processor, and a computer program stored in the memory and executable on the processor, where the processor executes the computer program to implement the method for monitoring DO in a biofilm or the oxygen supply optimization method based on image recognition described above.

According to specific embodiments provided in the present disclosure, the present disclosure achieves the following technical effects:

The present disclosure provides a method for monitoring DO in a biofilm, an oxygen supply optimization method and system based on image recognition, and a device. The monitoring method includes: acquiring image data of a biofilm in an MABR wastewater treatment apparatus during wastewater treatment; performing feature extraction on the image data to obtain image feature parameters; and inputting the image feature parameters into a biofilm DO prediction model to obtain an effective DO concentration within the biofilm outputted by the biofilm DO prediction model, where the biofilm DO prediction model is trained based on a machine learning model. This application provides a method for monitoring DO in a biofilm using machine learning-based image recognition, overcoming the drawbacks of microelectrodes such as high cost, susceptibility to damage, and difficult maintenance, thereby achieving low-cost and effective monitoring of DO in biofilms.

### BRIEF DESCRIPTION OF THE DRAWINGS

To describe the technical solutions in the embodiments of the present disclosure or in the prior art more clearly, the following briefly describes the accompanying drawings required for the embodiments. Apparently, the accompanying drawings in the following description show merely some embodiments of the present disclosure, and a person of ordinary skill in the art may still derive other accompanying drawings from these accompanying drawings without creative efforts.

FIG. 1 is a schematic flowchart of a method for monitoring DO in a biofilm according to an embodiment of the present disclosure;

FIG. 2 is a schematic flowchart of an oxygen supply optimization method based on image recognition according to an embodiment of the present disclosure;

FIG. 3 is a schematic structural diagram of an oxygen supply optimization system based on image recognition according to an embodiment of the present disclosure; and

FIG. 4 is a schematic structural diagram of a computer device according to an embodiment of the present disclosure.

Reference numerals: 1. Inlet tank; 2. Gas delivery unit; 201. Gas supply device; 202. Gas flow meter; 203. Pressure gauge; 3. Biochemical reaction unit; 301. Heterogeneous mass transfer biofilm membrane module; 302. Image recognition unit; 303. DO monitoring apparatus; 4. Intelligent terminal; 5. Outlet tank; 6. Peristaltic pump.

### DETAILED DESCRIPTION OF THE EMBODIMENTS

The technical solutions in the embodiments of the present disclosure are clearly and completely described below with reference to the accompanying drawings in the embodiments of the present disclosure. Apparently, the described embodiments are merely some rather than all of the embodiments of the present disclosure. All other embodiments obtained by a person of ordinary skill in the art based on the embodiments of the present disclosure without creative efforts shall fall within the protection scope of the present disclosure.

To make the above objectives, features, and advantages of the present disclosure more obvious and easy to understand, the present disclosure will be further described in detail with reference to the accompanying drawings and specific implementations.

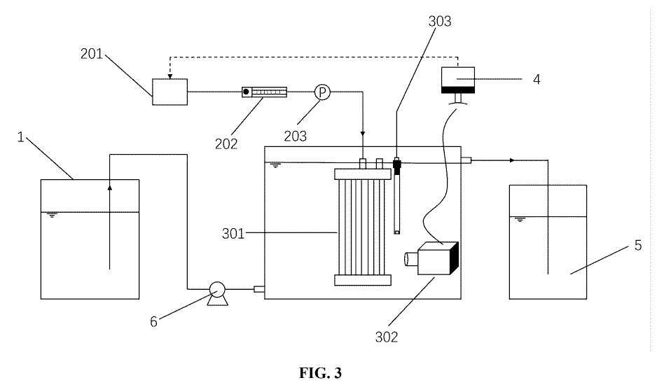

A method for monitoring DO in a biofilm, an oxygen supply optimization method and system, and a device of the present disclosure are all applied to an MABR wastewater treatment apparatus. As shown in FIG. 3, the MABR wastewater treatment apparatus includes: an inlet tank 1, an outlet tank 5, a gas delivery unit 2, and a biochemical reaction unit 3.

An inlet of the biochemical reaction unit 3 is connected to the inlet tank 1, with a peristaltic pump 6 arranged between the inlet of the biochemical reaction unit 3 and the inlet tank 1. An outlet of the biochemical reaction unit 3 is connected to the outlet tank 5. A heterogeneous mass transfer biofilm membrane module 301 is arranged in the biochemical reaction unit 3, where the heterogeneous mass transfer biofilm membrane module 301 is oriented perpendicular to a water flow direction.

The gas delivery unit 2 is connected to a gas inlet of the heterogeneous mass transfer biofilm membrane module 301 via a gas pipeline, with a gas flow meter 202 and a pressure gauge 203 installed in between. The gas flow meter 202 is used to regulate and monitor a gas flow rate, while the pressure gauge 203 monitors an aeration pressure.

A working principle of the MABR wastewater treatment apparatus is as follows:

Wastewater containing organic matter and ammonia nitrogen from the inlet tank is pumped into the biofilm reaction unit via the peristaltic pump. After a designated retention time, the treated water is discharged from the outlet to the outlet tank.

Prior to the formal operation of the gas delivery unit 2, biofilm inoculation needs to be completed. This involves adding a specific amount of nitrifying sludge into the biochemical reaction unit 3 while introducing ammonia nitrogen-containing wastewater to facilitate formation of a nitrifying biofilm on a surface of the heterogeneous mass transfer biofilm membrane module 301. The appearance of a yellowish-brown biofilm layer on the surface indicates successful inoculation.

During wastewater treatment, wastewater from the inlet tank 1 flows into the biochemical reaction unit 3, where a pollutant monitor measures influent pollutant concentrations. After the wastewater enters the biochemical reaction unit 3, the gas delivery unit 2 supplies gas to the heterogeneous mass transfer biofilm membrane module 301 through the pipeline. The gas is transferred to the biofilm through membrane fibers. Due to the attenuation of oxygen concentration during gas transfer, microorganisms at varying distances from the surface of the heterogeneous mass transfer biofilm membrane module 301 exhibit distinct pollutant removal characteristics. For instance, an inner layer hosts aerobic bacteria (nitrifying bacteria), while an outer layer contains anaerobic (facultative) bacteria (denitrifying bacteria), thereby achieving wastewater purification. The treated water is then discharged via the outlet to the outlet tank 5. The outlet tank 5 is equipped with a pollutant concentration detector.

The heterogeneous mass transfer biofilm membrane module includes a biofilm and an MABR membrane module, with the biofilm attached to the MABR membrane module.

As pollutant levels and biofilm conditions vary, the DO concentration in the biofilm changes accordingly. By employing image recognition technology, biofilm characteristics can be obtained to facilitate oxygen level regulation. This forms the basis for the method for monitoring DO in a biofilm, the oxygen supply optimization method and system, and the device of the present disclosure.

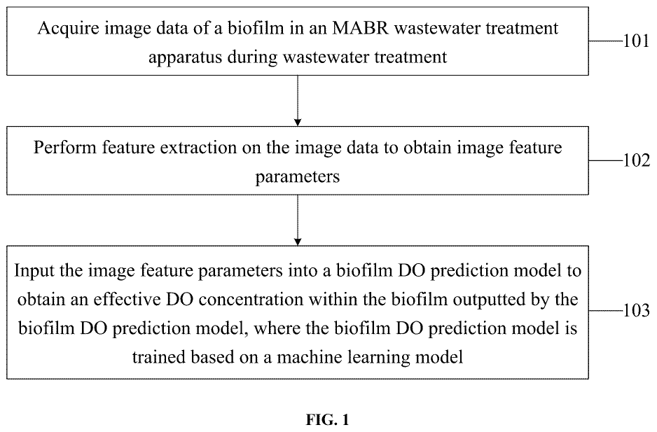

In one exemplary embodiment, as shown in FIG. 1, a method for monitoring DO in a biofilm is provided, including the following steps 101 to 103.

Step 101: Acquire image data of a biofilm in an MABR wastewater treatment apparatus during wastewater treatment.

Step 102: Perform feature extraction on the image data to obtain image feature parameters.

Step 103: Input the image feature parameters into a biofilm DO prediction model to obtain an effective DO concentration within the biofilm outputted by the biofilm DO prediction model, where the biofilm DO prediction model is trained based on a machine learning model.

By implementing steps 101 to 103, cost-effective and efficient monitoring of DO in the biofilm can be achieved.

In another exemplary embodiment, step 102 employs a deep learning algorithm for image feature extraction. Extracted image feature parameters, along with biofilm DO parameters, are input into the biofilm DO prediction model. The image feature parameters include biofilm surface bubble density, bubble size, biofilm thickness, biofilm color, and biofilm temperature distribution. The deep learning algorithm is constructed based on the OpenCV library.

In another exemplary embodiment, the biofilm DO prediction model in step 103 is obtained by training and optimizing a machine learning model according to predefined evaluation metrics. The machine learning model includes an Ant Colony Optimization (ACO)-Back Propagation (BP) neural network.

In another exemplary embodiment, the biofilm DO prediction model in step 103 requires periodic validation of prediction accuracy based on evaluation metrics.

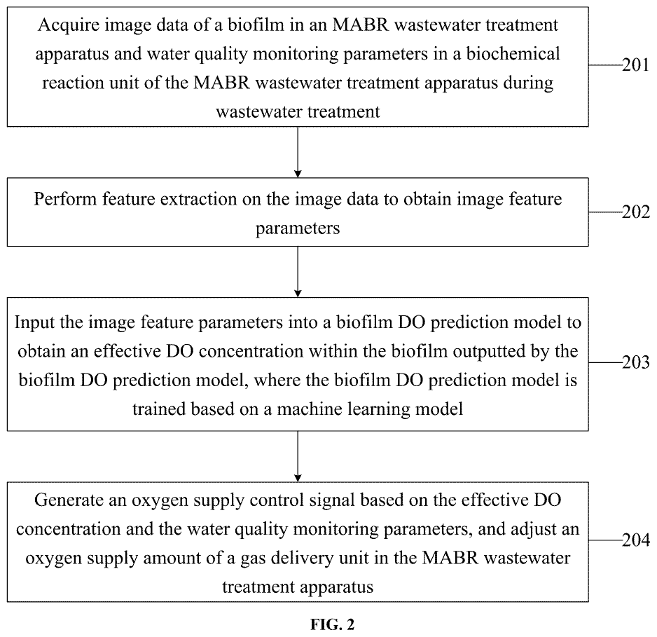

In one exemplary embodiment, as shown in FIG. 2, an oxygen supply optimization method based on image recognition is provided, including the following steps 201 to 204.

Step 201: Acquire image data of a biofilm in an MABR wastewater treatment apparatus and water quality monitoring parameters in a biochemical reaction unit of the MABR wastewater treatment apparatus during wastewater treatment.

Step 202: Perform feature extraction on the image data to obtain image feature parameters.

Step 203: Input the image feature parameters into a biofilm DO prediction model to obtain an effective DO concentration within the biofilm outputted by the biofilm DO prediction model, where the biofilm DO prediction model is trained based on a machine learning model.

Step 204: Generate an oxygen supply control signal based on the effective DO concentration and the water quality monitoring parameters, and adjust an oxygen supply amount of a gas delivery unit in the MABR wastewater treatment apparatus.

In another exemplary embodiment, the water quality monitoring parameters in step 201 include: influent ammonia nitrogen, effluent ammonia nitrogen, total nitrogen, COD concentration, and DO concentration in the biochemical reaction unit.

In another exemplary embodiment, in step 201, the image data is further screened with clearly captured areas cropped out, and image dimensions, contrast and brightness are adjusted until uniformity is achieved.

In another exemplary embodiment, a specific method for extracting the image feature parameters in step 202 includes:
under a Python environment with an OpenCV library installed, first using cv2.imread( ) to read an image, performing Gaussian filtering and edge detection preprocessing on the image, and calculating a pixel quantity to obtain biofilm color information; performing fitting on edge contours to derive biofilm thickness based on a minimum enclosing rectangle of biofilm contours; performing secondary processing on the preprocessed image, including grayscale conversion, binarization and background removal, and applying distance transform or erosion/dilation operations to isolate and count potential microbubbles on the surface of the biofilm.

Thermal imaging data of the biofilm is acquired through an infrared detector, with OpenCV employed to visualize temperature distribution characteristics, followed by normalization of thermal point values to obtain biofilm thermodynamic parameters.

In another exemplary embodiment, a training process in step 203 is as follows:

Obtained biofilm image parameters, thermodynamic parameters, ammonia nitrogen, total nitrogen, and COD measured in the inlet tank and outlet tank, solution DO in the biochemical reaction unit, and biofilm DO parameters measured by microelectrodes are inputted into an ACO-BP artificial neural network model, where a model dataset is divided into a training set and a test set, and the dataset incorporates laboratory measurements, network open-source databases and published research data. Further, model performance is evaluated using a mean square error, a mean absolute error, and a coefficient of determination, and hyperparameters are adjusted to optimize model fitting.

In another exemplary embodiment, a specific implementation of step 203 and step 204 is as follows:

Periodically acquired biofilm image parameters, thermodynamic parameters, ammonia nitrogen, total nitrogen, and COD measured in the inlet tank and outlet tank, solution DO in the biochemical reaction unit, and biofilm DO parameters measured by the microelectrodes are inputted as input datasets into the optimized ACO-BP model to output predicted effective DO concentration values in biofilm.

The prediction results are transmitted via data cable to a gas generation system, which selects an adjustment mode-primarily including output power adjustment and aeration mode adjustment-based on DO guidance values for specific biochemical reactions, thereby regulating a blower aeration rate to rectify a current biofilm DO level.

In another exemplary embodiment, in step 204, control measures of the gas delivery unit are adjusted according to the effective DO concentration in the biofilm and collected water quality monitoring parameters to complete the whole oxygen distribution optimization process. The adjusting the oxygen supply amount of the gas delivery unit in the MABR wastewater treatment apparatus includes one or more of: adjusting an aeration pressure, adjusting a gas flow rate, and adjusting an aeration mode.

The oxygen supply optimization method based on image recognition described in the above embodiments of the present disclosure introduces image recognition and a machine learning module to predict DO levels in heterogeneous mass transfer biofilms, providing a novel optimization approach for DO regulation in biofilm treatment. Compared with existing technologies, the present disclosure offers the following advantages:
1. Improved treatment efficiency or cost-effectiveness: In a heterogeneous mass transfer biofilm reactor, shifting the DO monitoring task from the solution to the biofilm enables guidance for precise aeration control, effectively preventing both insufficient and excessive aeration.
2. Intelligent decision-making support for biofilm DO monitoring: By inputting real-time monitoring data into an AI model, decision parameters can be obtained, making the water treatment process more accurate and capable of rapid responses to real-time water quality changes.
3. High scalability and adaptability to various scenarios: The optimization method of the present disclosure can be updated using a machine learning model.

In an exemplary embodiment, an oxygen supply optimization system based on image recognition is provided, as shown in FIG. 3. The system includes an oxygen supply adjustment apparatus, an image recognition unit 302, a DO monitoring apparatus 303, and an intelligent terminal 4. The oxygen supply adjustment apparatus is disposed between the gas delivery unit 2 of the MABR wastewater treatment apparatus and the heterogeneous mass transfer biofilm membrane module 301. The image recognition unit 302 and the DO monitoring apparatus 303 are arranged within the biochemical reaction unit 3 of the MABR wastewater treatment apparatus. Both the image recognition unit 302 and the DO monitoring apparatus 303 are connected to the intelligent terminal 4. The intelligent terminal 4 is connected to a control end of the oxygen supply adjustment apparatus. The image recognition unit 302 is configured to acquire image data of a biofilm in the heterogeneous mass transfer biofilm membrane module, while the DO monitoring apparatus is configured to acquire water quality monitoring parameters. The intelligent terminal 4 is configured to obtain an oxygen supply control signal based on the image data and the water quality monitoring parameters by using the above oxygen supply optimization method based on image recognition, and control the oxygen supply adjustment apparatus according to the oxygen supply control signal to regulate oxygen distribution.

In another exemplary embodiment, the image recognition unit 302 includes one or more of: an underwater camera, a laser scanning imager, and an infrared camera. In another exemplary embodiment, the image recognition unit 302 is located 30 mm to 1000 mm from the heterogeneous mass transfer biofilm module, while the DO monitoring apparatus is located 10 mm to 30 mm from the heterogeneous mass transfer biofilm module. The heterogeneous mass transfer biofilm module includes a biofilm and an MABR membrane module, with the biofilm attached to the MABR membrane module, the biofilm having a thickness exceeding 100 micrometers. Gas delivered by the gas delivery unit 2 is air or oxygen.

In another exemplary embodiment, the oxygen supply adjustment apparatus includes a membrane gas supply pressure monitoring component and a flow rate adjustment component, enabling adjustments to an aeration pressure, a gas flow rate, and an aeration mode to regulate oxygen distribution.

In the oxygen supply optimization system of the present disclosure, during treatment, the image recognition unit records real-time images of the biofilm, and a deep learning algorithm is employed to extract image features. Extracted image feature parameters and biofilm DO parameters are inputted into a machine learning model, which is trained and optimized based on predefined evaluation metrics to create a biofilm DO prediction model. Furthermore, based on a predicted effective DO concentration value in the biofilm and collected measured water quality data, control measures for the gas delivery unit are adjusted to complete the whole oxygen distribution optimization process.

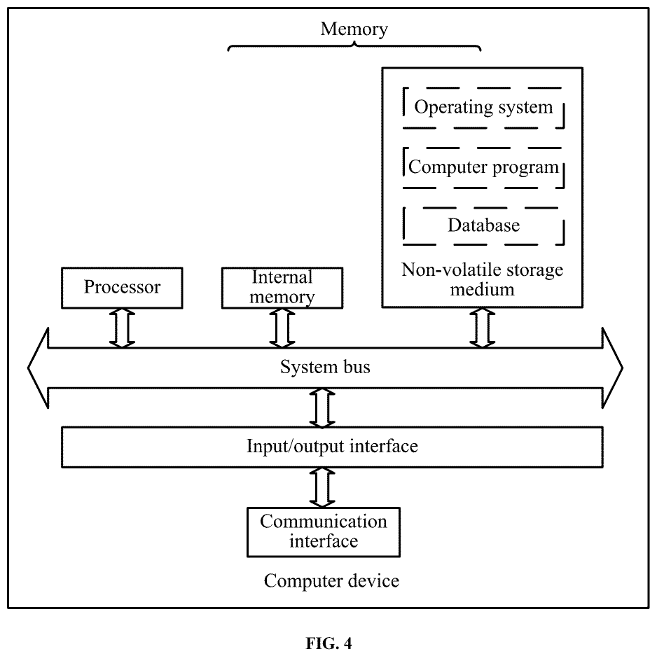

In an exemplary embodiment, a computer device is provided. The computer device may be a server or a terminal, and an internal structure thereof may be as shown in FIG. 4. The computer device includes a processor, a memory, an input/output (I/O) interface and a communication interface. The processor, the memory and the I/O interface are connected through a system bus. The communication interface is connected to the system bus through the I/O interface. The processor of the computer device is configured to provide computing and control capabilities. The memory of the computer device includes a non-volatile storage medium, and an internal memory. The non-volatile storage medium stores an operating system, a computer program, and a database. The internal memory provides an environment for operation of the operating system and the computer program in the non-volatile storage medium. The I/O interface of the computer device is configured to exchange information between the processor and an external device. The communication interface of the computer device is configured to communicate with an external terminal through a network. When executed by the processor, the computer program implements the method for monitoring DO in a biofilm or the oxygen supply optimization method based on image recognition described above.

Those skilled in the art may understand that the structure shown in FIG. 4 is only a block diagram of a part of the structure related to the solutions of the present disclosure and does not constitute a limitation on a computer device to which the solutions of the present disclosure are applied. Specifically, the computer device may include more or fewer components than those shown in the figure, or combine some components, or have different component arrangements. In an exemplary embodiment, a computer device is provided, including a memory and a processor, where the memory stores a computer program, and the computer program is executed by the processor to implement the steps of the above method embodiments.

To demonstrate the effectiveness of the method, system, and device in the aforementioned embodiments, the present disclosure provides the following specific examples.

A simulated wastewater with an ammonia nitrogen concentration of 50 mg/L was prepared in the inlet tank as the influent. The simulated wastewater contained 1 g/L sodium bicarbonate, 0.2 g/L glucose, and 1 mL/L trace elements. A pH value of the simulated wastewater ranges from 7 to 7.5.

Two identical biochemical reaction units were used for comparison: One unit employed the oxygen supply optimization method of the present disclosure (referred to as R1 hereinafter), and the other unit employed a conventional DO control method (referred to as R2 hereinafter). Both biochemical reaction units were inoculated with nitrifying sludge from an aerobic tank of a wastewater treatment plant, where Mixed Liquor Volatile Suspended Solids (MLVSS)=10 g/L. A hydraulic retention time was maintained at 24 h, with DO levels of 1-2 mg/L, until preliminary inoculation of nitrifying biofilm was achieved. During this phase, effluent pollutant levels stabilized, with ammonia nitrogen removal rates of 80-90% and total nitrogen removal rates consistently around 40% in this embodiment.

Comparative Example 1: An artificial wastewater with an ammonia nitrogen concentration of approximately 30 mg/L and a COD concentration of approximately 100 mg/L was prepared in the inlet tank. An aeration pressure of the gas generation device was set at 5 kPa, resulting in an ammonia nitrogen concentration of approximately 0 and a total nitrogen concentration of approximately 15 mg/L in effluent. A DO concentration in the biochemical reaction unit was approximately 2 mg/L. Based on the oxygen demand for nitrification, no adjustment was made to the gas supply mode of the R2 membrane module. For R1, the optimized ACO-BP model predicted a biofilm DO concentration of 4.5 mg/L, and an optimized aeration pressure was 1 kPa. After optimization, the R1 effluent had an ammonia nitrogen concentration of approximately 0 mg/L and a total nitrogen concentration of approximately 8 mg/L, while achieving 80% energy savings in gas generation.

Comparative Example 2: An artificial wastewater with an ammonia nitrogen concentration of approximately 100mg/L and a COD concentration of approximately 200 mg/L was prepared in the inlet tank. An aeration pressure of the gas generation device was set at 2 kPa, resulting in an ammonia nitrogen concentration of approximately 30 mg/L and a total nitrogen concentration of approximately 70 mg/L in effluent. A DO concentration in the biochemical reaction unit was approximately 0 mg/L. Based on the oxygen demand for nitrification, the aeration pressure was adjusted to 4 kPa to achieve a DO concentration of 2 mg/L in R2. After adjustment, the ammonia nitrogen concentration was 5 mg/L and the total nitrogen concentration was about 70 mg/L in effluent. For R 1, the optimized ACO-BP model predicted a biofilm DO concentration of 1.5 mg/L, and an optimized aeration pressure was 3 kPa. After optimization, the R1 effluent had an ammonia nitrogen concentration of approximately 10 mg/L and a total nitrogen concentration of approximately 60 mg/L, while achieving 25% energy savings in gas generation.

It should be noted that during use, the ACO-BP model embedded in the intelligent terminal can be replaced later, and the built-in artificial neural network model of the platform also requires periodic calibration. That is, after simulation and verification, the artificial neural network model needs to be regularly tested for reliability during operation. If accuracy decreases, the model needs to be replaced or retrained.

It is to be noted that information of a user (including but not limited to device information of the user, personal information of the user and the like) and data (including but not limited to data for analysis, data for storage, data for exhibition and the like) in the present disclosure are information and data authorized by the user or fully authorized by each party.

Those of ordinary skill in the art may understand that all or some of the procedures in the method of the foregoing embodiments may be implemented by a computer program instructing related hardware. The computer program may be stored in a non-volatile computer-readable storage medium. When the computer program is executed, the procedures in the embodiments of the foregoing method may be performed. Any reference to a memory, a database, or other media used in the embodiments of the present application may include a non-volatile and/or volatile memory. The non-volatile memory may include a read-only memory (ROM), a magnetic tape, a floppy disk, a flash memory, an optical memory, a high-density embedded nonvolatile memory, a resistive random access memory (ReRAM), a magnetoresistive random access memory (MRAM), a ferroelectric random access memory (FRAM), a phase change memory (PCM), a graphene memory, etc. The volatile memory may include a random access memory (RAM) or an external cache memory. As an illustration rather than a limitation, the RAM may be in various forms, such as a static random access memory (SRAM) or a dynamic random access memory (DRAM).

The database in the embodiments of the present disclosure may include at least one of a relational database and a non-relational database. The non-relational database may include a distributed database based on a blockchain, but is not limited thereto. The processor in the embodiments of the present disclosure may be a general processor, a central processor, a graphics processor, a digital signal processor (DSP), a programmable logic device, and a data processing logic device based on quantum computing, but is not limited thereto.

The technical features of the above embodiments can be employed in arbitrary combinations. To provide a concise description of these embodiments, all possible combinations of all the technical features of the above embodiments may not be described; however, these combinations of the technical features should be construed as falling within the scope defined by the specification as long as no contradiction occurs.

Several examples are used herein for illustration of the principles and implementations of the present disclosure. The description of the foregoing examples is used to help illustrate the method of the present disclosure and the core principles thereof. In addition, those of ordinary skill in the art can make various modifications in terms of specific implementations and scope of application in accordance with the teachings of the present disclosure. In conclusion, the content of the present specification shall not be construed as a limitation to this application.

What is claimed is:

1. A method for monitoring dissolved oxygen (DO) in a biofilm, wherein the method for monitoring DO in a biofilm is applied to a Membrane Aerated Biofilm Reactor (MABR) wastewater treatment apparatus, and comprises:
acquiring image data of a biofilm in the MABR wastewater treatment apparatus during wastewater treatment;
performing feature extraction on the image data to obtain image feature parameters; and
inputting the image feature parameters into a biofilm DO prediction model to obtain an effective DO concentration within the biofilm outputted by the biofilm DO prediction model, wherein the biofilm DO prediction model is trained based on a machine learning model.

2. The method for monitoring DO in a biofilm according to claim 1, wherein the image feature parameters comprise: a biofilm surface bubble density, a surface bubble size, a biofilm thickness, a biofilm color, and a biofilm temperature distribution.

3. An oxygen supply optimization method based on image recognition, wherein the oxygen supply optimization method based on image recognition is applied to a Membrane Aerated Biofilm Reactor (MABR) wastewater treatment apparatus, and comprises:
acquiring image data of a biofilm in the MABR wastewater treatment apparatus and water quality monitoring parameters in a biochemical reaction unit of the MABR wastewater treatment apparatus during wastewater treatment;
performing feature extraction on the image data to obtain image feature parameters;
inputting the image feature parameters into a biofilm dissolved oxygen (DO) prediction model to obtain an effective DO concentration within the biofilm outputted by the biofilm DO prediction model, wherein the biofilm DO prediction model is trained based on a machine learning model; and
generating an oxygen supply control signal based on the effective DO concentration and the water quality monitoring parameters, and adjusting an oxygen supply amount of a gas delivery unit in the MABR wastewater treatment apparatus.

4. The oxygen supply optimization method based on image recognition according to claim 3, wherein the water quality monitoring parameters comprise: an influent ammonia nitrogen, an effluent ammonia nitrogen, a total nitrogen, a chemical oxygen demand (COD) concentration, and a DO concentration in the biochemical reaction unit; and
the image feature parameters comprise: a biofilm surface bubble density, a surface bubble size, a biofilm thickness, a biofilm color, and a biofilm temperature distribution.

5. The oxygen supply optimization method based on image recognition according to claim 3, wherein the adjusting the oxygen supply amount of the gas delivery unit in the MABR wastewater treatment apparatus comprises one or more of: adjusting an aeration pressure, adjusting a gas flow rate, and adjusting an aeration mode.

6. An oxygen supply optimization system based on image recognition, wherein the oxygen supply optimization system based on image recognition is applied to a Membrane Aerated Biofilm Reactor (MABR) wastewater treatment apparatus, and comprises:
an oxygen supply adjustment apparatus, an image recognition unit, a dissolved oxygen (DO) monitoring apparatus, and an intelligent terminal;
wherein the oxygen supply adjustment apparatus is disposed between a gas delivery unit of the MABR wastewater treatment apparatus and a heterogeneous mass transfer biofilm membrane module;
wherein the image recognition unit and the DO monitoring apparatus are arranged in a biochemical reaction unit of the MABR wastewater treatment apparatus;
wherein the image recognition unit and the DO monitoring apparatus are both connected to the intelligent terminal, and the intelligent terminal is connected to a control end of the oxygen supply adjustment apparatus;
wherein the image recognition unit is configured to acquire image data of a biofilm in the heterogeneous mass transfer biofilm membrane module, and the DO monitoring apparatus is configured to obtain water quality monitoring parameters; and
wherein the intelligent terminal is configured to
obtain an oxygen supply control signal based on the image data and the water quality monitoring parameters by:
performing feature extraction on the image data to obtain image feature parameters;
inputting the image feature parameters into a biofilm DO prediction model to obtain an effective DO concentration within the biofilm outputted by the biofilm DO prediction model, wherein the biofilm DO prediction model is trained based on a machine learning model; and
generating the oxygen supply control signal based on the effective DO concentration and the water quality monitoring parameters, and
control the oxygen supply adjustment apparatus according to the oxygen supply control signal to regulate oxygen distribution.

7. The oxygen supply optimization system based on image recognition according to claim 6, wherein the oxygen supply adjustment apparatus comprises one or both of a membrane gas supply pressure monitoring component and a flow rate adjustment component.

8. The oxygen supply optimization system based on image recognition according to claim 6, wherein the image data is acquired by one or more of: an underwater camera, a laser scanning imager, and an infrared camera.

9. The oxygen supply optimization system based on image recognition according to claim 6, wherein a distance between the image recognition unit and the heterogeneous mass transfer biofilm membrane module is 30 mm to 1000 mm; and a distance between the DO monitoring apparatus and the heterogeneous mass transfer biofilm membrane module is 10 mm to 30 mm.

10. The oxygen supply optimization system based on image recognition according to claim 6, wherein the water quality monitoring parameters comprise: influent ammonia nitrogen, effluent ammonia nitrogen, total nitrogen, chemical oxygen demand (COD) concentration, and DO concentration in the biochemical reaction unit; and
the image feature parameters comprise: biofilm surface bubble density, surface bubble size, biofilm thickness, biofilm color, and biofilm temperature distribution.

11. The oxygen supply optimization system based on image recognition according to claim 6, wherein the adjusting the oxygen supply amount of the gas delivery unit in the MABR wastewater treatment apparatus comprises one or more of: adjusting an aeration pressure, adjusting a gas flow rate, and adjusting an aeration mode.

12. A computer device, comprising a memory, a processor, and a computer program stored in the memory and executable on the processor, wherein the processor executes the computer program to:
acquire image data of a biofilm in a Membrane Aerated Biofilm Reactor (MABR) wastewater treatment apparatus during wastewater treatment;
perform feature extraction on the image data to obtain image feature parameters; and
input the image feature parameters into a biofilm dissolved oxygen (DO) prediction model to obtain an effective DO concentration within the biofilm outputted by the biofilm DO prediction model, wherein the biofilm DO prediction model is trained based on a machine learning model.

13. The computer device according to claim 12, wherein the image feature parameters comprise: a biofilm surface bubble density, a surface bubble size, a biofilm thickness, a biofilm color, and a biofilm temperature distribution.

14. A computer device, comprising a memory, a processor, and a computer program stored in the memory and executable on the processor, wherein the processor executes the computer program to:
acquire image data of a biofilm in a Membrane Aerated Biofilm Reactor (MABR) wastewater treatment apparatus and water quality monitoring parameters in a biochemical reaction unit of the MABR wastewater treatment apparatus during wastewater treatment;
perform feature extraction on the image data to obtain image feature parameters;
input the image feature parameters into a biofilm dissolved oxygen (DO) prediction model to obtain an effective DO concentration within the biofilm outputted by the biofilm DO prediction model, wherein the biofilm DO prediction model is trained based on a machine learning model; and
generate an oxygen supply control signal based on the effective DO concentration and the water quality monitoring parameters, and adjust an oxygen supply amount of a gas delivery unit in the MABR wastewater treatment apparatus.

15. The computer device according to claim 14, wherein the water quality monitoring parameters comprise: an influent ammonia nitrogen, an effluent ammonia nitrogen, a total nitrogen, a chemical oxygen demand (COD) concentration, and a DO concentration in the biochemical reaction unit; and
the image feature parameters comprise: a biofilm surface bubble density, a surface bubble size, a biofilm thickness, a biofilm color, and a biofilm temperature distribution.

16. The computer device according to claim 14, wherein the adjusting the oxygen supply amount of the gas delivery unit in the MABR wastewater treatment apparatus comprises one or more of: adjusting an aeration pressure, adjusting a gas flow rate, and adjusting an aeration mode.
